(19) United States
(12) Patent Application Publication
(10) Pub. No.: US 2023/0092532 A1
(43) Pub. Date: Mar. 23, 2023

(54) METHOD TO PREVENT SAMPLE PREPARATION-INDUCED DISULFIDE SCRAMBLING IN NON-REDUCED PEPTIDE MAPPING

(71) Applicant: Regeneron Pharmaceuticals, Inc, Tarrytown, NY (US)

(72) Inventors: Song Nie, Armonk, NY (US); Tyler Greer, Elmsford, NY (US); Xiaojing Zheng, Gaithersburg, MD (US)

(21) Appl. No.: 17/945,398

(22) Filed: Sep. 15, 2022

Related U.S. Application Data
(60) Provisional application No. 63/245,565, filed on Sep. 17, 2021.

(51) Int. Cl. G01N 33/68; C07K 1/16

(52) U.S. Cl. CPC G01N 33/6857; G01N 33/6842; C07K 1/16

(57) ABSTRACT

The present invention generally pertains to methods of preventing disulfide scrambling in non-reducing liquid chromatography-mass spectrometry analysis of a protein of interest. In particular, the present invention pertains to the addition of cystamine to a non-reducing liquid chromatography-mass spectrometry analysis of an antibody to prevent disulfide scrambling.

### CROSS-REFERENCE TO RELATED APPLICATIONS

This application claims priority to and the benefit of U.S. Provisional Patent Application No. 63/245,565, filed Sep. 17, 2021, which is herein incorporated by reference.

### FIELD

This application relates to methods for characterization of disulfide bonds in a protein of interest.

### BACKGROUND

Characterization of monoclonal antibodies' (mAbs) product quality attributes (PQAs) is important due to the large size and complex heterogeneity of this major class of therapeutics. One such PQA is the proper formation of classical disulfide bond structures. Deviations from the canonical IgG disulfide conformation, including non-classical disulfide bonding (scrambling), may negatively impact a mAb's structure, stability, and biological efficacy.

Liquid chromatography-mass spectrometry (LC-MS) is a powerful method for in-depth profiling of mAb PQAs, including canonical disulfide bond formation and identification of non-classical disulfide features like disulfide bond scrambling. The most common LC-MS approach to study mAb disulfide bonds is non-reduced peptide mapping. This method involves enzymatically digesting a mAb into peptide species, with any potential disulfide bonds remaining intact. Peptides are then analyzed by LC-MS, where a UV detector generates a “peptide fingerprint” by measuring UV absorbance of the eluting analytes according to their retention times, and a mass spectrometer ionizes these analytes and records their mass-to-charge ratios (m/z).

However, experimental conditions and reagents used in non-reduced peptide mapping, such as heating or alkaline pH, can potentially induce the formation of scrambled disulfide artifacts, confounding the interpretation of native disulfide bond scrambling. To reduce disulfide scrambling artifacts induced during sample preparation, several strategies have been developed. One method is to alkylate free cysteines with an excess amount of iodoacetamide; however, this method does not prevent disulfide disruption and may cause nonspecific labeling of other residues. Another strategy is the use of acidic pH conditions during denaturation and digestion, using N-ethylmaleimide (NEM) as an alkylation agent and a digestive enzyme such as pepsin, but this method may result in non-specific ragged cleavage and an interfering UV signal from NEM. A third approach is the use of rLys-C and trypsin at acidic pH to efficiently cleave arginine and lysine residues while minimizing scrambling; however, this method also may result in inferior digestion specificity and efficiency, which can interfere with accurate analysis of disulfide bonds.

Therefore, demand exists for methods and systems to prevent disulfide bond scrambling in non-reduced peptide mapping analysis that are simple and allow for sensitive and specific characterization of native disulfide bonds.

### SUMMARY

A method has been developed for non-reduced peptide mapping to characterize disulfide bonds of a protein of interest, while preventing the formation of sample preparation-induced disulfide scrambling. The method includes the novel addition of cystamine during sample preparation to prevent native disulfide disruption. This cystamine-added non-reduced peptide mapping method allows for sample preparation at an alkaline pH, and may allow for protein denaturation at high temperatures without inducing disulfide scrambling.

This disclosure provides a method for characterizing at least one disulfide bond of a protein of interest. In some exemplary embodiments, the method comprises (a) preparing a peptide digest of a protein of interest, said preparing including: (i) contacting a sample including a protein of interest to cystamine and to at least one denaturation agent to form a denatured protein of interest; (ii) contacting said denatured protein of interest to an alkylation agent to form an alkylated protein of interest; and (iii) contacting said alkylated protein of interest to a digestive enzyme to form a peptide digest; (b) subjecting said peptide digest to analysis using liquid chromatography-mass spectrometry to identify at least one peptide that includes a disulfide bond; and (c) using said at least one identified peptide to characterize at least one disulfide bond of said protein of interest.

In one aspect, the method further comprises adding cystamine to said denatured protein of interest, adding cystamine to said alkylated protein of interest, or a combination thereof. In another aspect, the concentration of cystamine is between about 0.5 mM and about 2 mM, optionally wherein the concentration of cystamine is about 1 mM.

In one aspect, the method further comprises comparing said at least one identified peptide to at least one identified peptide from a control sample including said protein of interest, wherein said control sample is additionally subjected to a protein reduction step.

In one aspect, said protein of interest is an antibody. In a specific aspect, said protein of interest is a monoclonal antibody or a bispecific antibody.

In one aspect, said at least one denaturation agent is urea. In a specific aspect, said urea is present at between about 6 M and about 10 M, optionally wherein said urea is present about 8 M. In another aspect, said denaturation is conducted at a pH between about 7 and about 8, optionally wherein said denaturation is conducted at a pH of about 7.5. In yet another aspect, said denaturation is conducted at about 37° C. or about 50° C.

In one aspect, said alkylation agent is iodoacetamide. In a specific aspect, said iodoacetamide is present at between about 1 mM and about 20 mM, optionally wherein said iodoacetamide is present at about 2.5 mM. In another aspect, said alkylation is conducted at a pH between about 7 and about 8, optionally wherein said alkylation is conducted at a pH of about 7.5.

In one aspect, said digestive enzyme is trypsin. In a specific aspect, said trypsin is present at between about a 1:5 enzyme:substrate ratio and about a 1:20 enzyme:substrate ratio, optionally wherein said trypsin is present at about a 1:10 enzyme:substrate ratio. In another aspect, said digestion is conducted at a pH between about 7 and about 8, optionally wherein said digestion is conducted at a pH of about 7.5.

In one aspect, said chromatography step comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1A illustrates a schematic of an IgG1 antibody with disulfide bonds indicated, according to an exemplary embodiment.

FIG. 1B shows identified disulfide scrambled peptides from non-reduced peptide mapping analysis of mAb1 according to an exemplary embodiment.

FIG. 2 shows extracted ion chromatograms of the LC4-LC5 disulfide scrambled peptide obtained from non-reduced peptide mapping analysis of mAb1 prepared in low pH and regular control conditions according to an exemplary embodiment.

FIG. 3A shows a UV chromatogram of non-reduced peptide mapping analysis of mAb1 prepared by the low pH method, with an NEM reagent interference peak labeled, according to an exemplary embodiment.

FIG. 3B shows UV chromatograms of non-reduced peptide mapping analysis of mAb1 using 2.5 mM iodoacetamide (IAA) or 100 mM IAA according to an exemplary embodiment.

FIG. 4A shows a normalized peak area of disulfide scrambled peptide LC4-LC5 from non-reduced peptide mapping analysis of mAb1 in five different experimental conditions according to an exemplary embodiment.

FIG. 4B shows a normalized peak area of disulfide scrambled peptide HC3-HC5 from non-reduced peptide mapping analysis of mAb1 in five different experimental conditions according to an exemplary embodiment.

FIG. 4C shows extracted ion chromatograms of disulfide scrambled peptide LC4-LC5 from non-reduced peptide mapping (NRPM) analysis of mAb1 in different experimental conditions according to an exemplary embodiment.

FIG. 5A shows a normalized peak area of disulfide scrambled peptide LC4-LC5 from non-reduced peptide mapping analysis of mAb2 in five different experimental conditions according to an exemplary embodiment.

FIG. 5B shows a normalized peak area of disulfide scrambled peptide HC3-HC5 from non-reduced peptide mapping analysis of mAb2 in five different experimental conditions according to an exemplary embodiment.

FIG. 5C shows extracted ion chromatograms of disulfide scrambled peptide LC4-LC5 from non-reduced peptide mapping analysis of mAb2 in different experimental conditions according to an exemplary embodiment.

FIG. 6A shows UV chromatograms from non-reduced peptide mapping analysis of mAb1 samples denatured and alkylated at different temperatures according to an exemplary embodiment.

FIG. 6B shows peak areas of native disulfide peptides from mAb1 using the regular and cystamine-added non-reduced peptide mapping methods at different temperatures according to an exemplary embodiment.

FIG. 6C shows peak areas of disulfide scrambled peptide LC4-LC5 from regular or cystamine-added non-reduced peptide mapping analysis of mAb2 samples denatured and alkylated at different temperatures according to an exemplary embodiment.

### DETAILED DESCRIPTION

Characterization of monoclonal antibodies' (mAbs) product quality attributes (PQAs) is important due to the large size and complex heterogeneity of this increasingly popular class of therapeutics. One such PQA is the proper formation of classical disulfide bond structures. Deviations from the canonical IgG disulfide conformation, including non-classical disulfide bonding (scrambling), may negatively impact a mAb's structure, stability, and biological efficacy (Zhang et al., 2011, Biotechnol Adv, 29(6):923-9; Liu et al., 2012, MAbs, 4(1):17-23; Liu et al., 2007, Biotechnol Lett, 29(11):611-22; Brych et al., 2010, J Pharm Sci, 99(2):764-81; Mamathambika and Bardwell, 2008, Annu Rev Cell Dev Biol, 24:211-35; Zhang et al., 2012, Anal Chem, 84(16):7112-23; Van Buren et al., 2009, J Pharm Sci, 98(9):3013-30; Zhang et al., 2019, Protein Expr Purif, 164:105459).

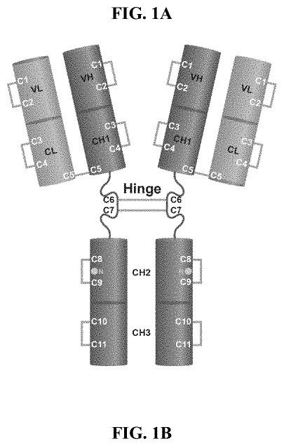

Disulfide bond conformation is highly conserved in accordance with each IgG subclass (Milstein, 1966, Biochem J, 101(2):338-51; Pinck and Milstein, 1967, Nature, 216(5118):941-2; Frangione and Milstein, 1968, J Mol Biol, 33(3):893-906; Frangione et al., 1969, Nature, 221(5176):145-8). For example, IgG1 molecules have a four-chain structure composed of two heavy chains (HCs) and two light chains (LCs) covalently linked by inter-chain disulfide bonds, as shown in FIG. 1A. In addition to the inter-chain disulfide bonds, one intra-chain disulfide bond is present and is shielded within each (3-barrel domain of the HC and LC polypeptides (Zhang et al., 2002, Anal Biochem, 311(1):1-9). In the hinge region, the two HCs are covalently linked by two inter-chain disulfide bonds.

A typical therapeutic mAb has a molecular weight of about 140 kDa, rendering traditional disulfide bond mapping methods, such as NMR (Klaus et al., 1993, J Mol Biol, 232(3):897-906), X-ray crystallography (Jones et al., 1997, Methods Enzymol, 277:173-208), and Edman sequencing (Haniu et al., 1994, Int J Pept Protein Res, 43(1):81-6) less applicable. The rapid evolution of liquid chromatography-mass spectrometry (LC-MS) and its successful implementation in biomolecule analysis has enabled in-depth profiling of mAb PQAs, including canonical disulfide bond formation and identification of non-classical disulfide features like disulfide bond scrambling, free thiol, and trisulfide bond formation. The most common LC-MS approach to study mAb disulfide bonds, known as non-reduced peptide mapping, is a modified version of the conventional reduced peptide mapping approach with no disulfide reduction step and lower amount of thiol alkylating agent (Li et al., 2015, State-of-the-Art and Emerging Technologies for Therapeutic Monoclonal Antibody Characterization Volume 2. Biopharmaceutical Characterization: The NISTmAb Case Study, pp. 119-183; Formolo et al., 2015, State-of-the-Art and Emerging Technologies for Therapeutic Monoclonal Antibody Characterization Volume 2. Biopharmaceutical Characterization: The NISTmAb Case Study, pp. 1-62). Trypsin is the most commonly used digestive enzyme due to its high specificity, efficiency, and propensity to generate peptides of appropriate length for MS analysis. The resulting method enzymatically cleaves the mAb into peptide species, with any potential disulfide bonds remaining intact. All peptides are then analyzed by LC-MS, where a UV detector generates a “peptide fingerprint” by measuring the UV absorbance of the eluting analytes according to their retention times, and a mass spectrometer ionizes these analytes and records their mass-to-charge ratios (m/z). High-resolution accurate-mass (HRAM) mass spectrometers with tandem mass spectrometry (MS²) capabilities coupled to advanced protein/peptide identification algorithms like Byonic have simplified peptide mapping analysis so that even sensitive identification of disulfide-linked peptides and site-specific identification of free thiol are now routine.

The high selectivity and sensitivity of non-reduced peptide mapping inherits a disadvantage associated with reduced peptide mapping: experimental conditions and reagents can sometimes induce confounding chemical modifications into peptide sequences if the method is not thoroughly optimized and carefully developed. For non-reduced peptide mapping, scrambled disulfide artifacts were found to be associated with sample preparation steps such as denaturation by heating and/or enzymatic digestion conditions at alkaline pH. These experimentally introduced scrambled disulfide artifacts may lead to false interpretations or conclusions regarding their pre-existing levels in the native therapeutic mAbs (Liu et al., 2007; Zhang et al., 2002, Anal Biochem, 311(1):1-9; Wu and Watson, 1997, Protein Sci, 6(2):391-8).

To reduce disulfide scrambling artifacts during non-reduced analyses, several strategies have been developed. The simplest approach is to alkylate free cysteine using an excess amount of iodoacetamide, which essentially caps all endogenous free thiols as well as artifact thiols before any scrambling can occur. However, this method fails to prevent undesired disulfide disruption, and a large excess of iodoacetamide causes nonspecific labeling of other residues that are sometimes visible in the UV chromatogram (Boja and Fales, 2001, Anal Chem, 73(15): 3576-82; Muller and Winter, 2017, Mol Cell Proteomics, 16(7):1173-1187).

Another strategy to minimize disulfide scrambling is to conduct denaturation and digestion at acidic pH while capping free thiol with N-ethylmaleimide (NEM) due to its high reactivity in acidic conditions (Ryle et al., 1955, Biochem J, 60(4):541-56; Robotham and Kelly, 2019, MAbs, 11(4):757-766). To circumvent the low activity of trypsin in acidic pH and bolster digestion efficiency, alternative enzymes like pepsin with acceptable activities at low pH have been used, but the non-specific ragged cleavages makes the assignment of disulfide bonds rather complex.

Another solution, pioneered by Promega™ and produced as a digestion kit called AccuMAP™, utilizes rLys-C and trypsin at acidic pH to efficiently cleave arginine and lysine residues while minimizing scrambling. However, digestion specificity and efficiency still suffer, and a one-enzyme approach that minimizes disulfide scrambling with the high digestion specificity and efficiency of trypsin is desirable to ensure assay reproducibility and robustness. In addition, such an approach would simplify the method development, qualification and, potential technical transfer steps in pharmaceutical companies.

The disclosure herein provides an elegant solution to prevent disulfide bonds in mAbs from scrambling at alkaline pH during non-reduced tryptic digestion conditions. A standard peptide mapping protocol was modified by adding the compound cystamine to the sample preparation buffer used to dilute the therapeutic mAb prior to denaturation and alkylation of native free thiols. Two in-house IgG1 mAbs were selected in this study because a relatively high level of scrambled disulfide bonds was identified in the samples when a conventional non-reduced peptide mapping protocol was implemented. These two mAbs were used to demonstrate that adding cystamine to non-reduced peptide mapping protocols eliminates disulfide scrambling artifacts even at alkaline pH and high denaturation temperatures. This new cystamine-added non-reduced peptide mapping method, with minimal changes to a standard protocol, enables confident analysis of disulfide connectivity and identification of disulfide scrambling while maintaining the advantages of tryptic digestion at alkaline pH. Furthermore, this technique can be applied as a platform method for mAb disulfide characterization at all stages of drug development.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

In some exemplary embodiments, the sample including the protein of interest can be prepared prior to LC/UV-MS analysis. Preparation steps can include denaturation, alkylation, dilution and digestion.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

As used herein, the term “protein reducing agent” or “reduction agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample containing the at least one protein of interest or peptide digest can be subjected to any one of the aforementioned chromatographic methods or a combination thereof.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS′), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. In some exemplary embodiments, disulfide bonds of the peptides identified by the mass spectrometer can be used as surrogate representatives of disulfide bonds of the intact protein. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, automated iterative MS/MS can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

This disclosure provides a method for characterizing at least one disulfide bond in a protein of interest. In some exemplary embodiments, the method comprises (a) preparing a peptide digest of a protein of interest, said preparing including: (i) contacting a sample including a protein of interest to cystamine and to at least one denaturation agent to form a denatured protein of interest; (ii) contacting said denatured protein of interest to an alkylation agent to form an alkylated protein of interest; and (iii) contacting said alkylated protein of interest to a digestive enzyme to form a peptide digest; (b) subjecting said peptide digest to analysis using liquid chromatography-mass spectrometry to identify at least one peptide that includes a disulfide bond; and (c) using said at least one identified peptide to characterize at least one disulfide bond of said protein of interest.

In some exemplary embodiments, the method of the present invention further comprises the addition of cystamine to the denatured protein of interest, the alkylated protein of interest, or both. It is desirable to maintain cystamine at a functional concentration throughout the sample preparation process in order to prevent disulfide scrambling. This may be accomplished through one-time addition of cystamine or by repeatedly adding cystamine throughout the sample preparation. A functional concentration of cystamine may be about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, or about 2 mM. A person skilled in the art can adjust the concentration of cystamine according to the particular needs of an experiment. In some exemplary embodiments, a functional concentration of cystamine may be about 1 mM.

Additional information about a protein of interest may be gathered by comparison to a control sample. A control sample may be, for example, a sample including the same protein of interest that is subjected to reduced peptide mapping instead of non-reduced peptide mapping. Peptides identified through reduced peptide mapping analysis of a control sample would be analogous to peptides identified through non-reduced peptide mapping analysis of the experimental sample except for the presence of disulfide bonds, allowing for a direct comparison between peptides generated with or without disulfide bonds. In some exemplary embodiments, a control sample may be subjected to reduced peptide mapping analysis by contacted said sample to a reducing agent, for example TCEP.

The method of the present invention may be applied to any protein featuring disulfide bonds. In some exemplary embodiments, a particular application involves analysis of a protein of interest that is an antibody. In some exemplary embodiments, the protein of interest is a monoclonal antibody. In some exemplary embodiments, the protein of interest is a bispecific antibody. In some exemplary embodiments, the protein of interest is a recombinant protein.

A variety of denaturation agents may be used in the sample preparation step of the method of the present invention, for example, guanidine hydrochloride or urea. In some exemplary embodiments, the denaturation agent is urea. Urea may be used at a concentration of about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, about 8 M, about 8.1 M, about 8.2 M, about 8.3 M, about 8.4 M, about 8.5 M, about 8.6 M, about 8.7 M, about 8.8 M, about 8.9 M, about 9 M, about 9.1 M, about 9.2 M, about 9.3 M, about 9.4 M, about 9.5 M, about 9.6 M, about 9.7 M, about 9.8 M, about 9.9 M, or about 10 M. In some exemplary embodiments, an optimal concentration of urea is about 8 M.

Denaturation may be conducted in a variety of conditions. Acidic pH conditions have been used to reduce disulfide scrambling. One of the advantages of the method of the present invention is the ability to reduce disulfide scrambling even at alkaline pH. Denaturation may be conducted at a pH of about 7, about 7.1, about 7.2, about 7.3, about 7.4, about 7.5, about 7.6, about 7.7, about 7.8, about 7.9, or about 8. In some exemplary embodiments, an optimal pH for denaturation is about 7.5.

The temperature at which denaturation is conducted may impact disulfide scrambling and digestion efficiency. Lower temperatures, for example 37° C., may lead to less disulfide scrambling, but also lead to less effective denaturation, which in turn lowers digestion efficiency and may confound peptide mapping analysis. It may be desirable to subject a protein of interest to denaturation at a higher temperature, for example 50° C., in order to improve peptide mapping analysis, provided that the sample preparation protocol can prevent disulfide scrambling even at 50° C. The method of the present invention may successfully prevent disulfide scrambling even when conducting denaturation at 50° C. In some exemplary embodiments, the denaturation step is conducted at about 37° C. In some exemplary embodiments, the denaturation step is conducted at about 50° C. Denaturation may be conducted at about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C.

In some exemplary embodiments, the alkylation agent used is iodoacetamide (IAA). IAA can be used at a relatively wide range of concentrations. Higher concentrations of IAA are more effective at preventing disulfide scrambling, but may result in over-alkylation artifacts. One of the advantages of the method of the present invention is the ability to prevent disulfide scrambling even with a concentration of IAA within a conventional range that avoids over-alkylation artifacts. The concentration of IAA may be about 1 mM, about 1.1 mM about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, about 3.7 mM, about 3.8 mM, about 3.9 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM. In some exemplary embodiments, an optimal concentration of IAA is about 2.5 mM.

Alkylation may be conducted at a pH of about 7, about 7.1, about 7.2, about 7.3, about 7.4, about 7.5, about 7.6, about 7.7, about 7.8, about 7.9, or about 8. In some exemplary embodiments, an optimal pH for alkylation is about 7.5.

Digestive enzymes used for non-reduced peptide mapping may include, for example, trypsin, pepsin, or LysC. In some exemplary embodiments, the digestive enzyme is trypsin. Trypsin may be used at an enzyme:substrate ratio of about 1:5, about 1:5.5, about 1:6, about 1:6.5, about 1:7, about 1:7.5, about 1:8, about 1:8.5, about 1:9, about 1:9.5, about 1:10, about 1:10.5, about 1:11, about 1:11.5, about 1:12, about 1:12.5, about 1:13, about 1:13.5, about 1:14, about 1:14.5, about 1:15, about 1:16, about 1:17, about 1:18, about 1:19, or about 1:20. In some exemplary embodiments, an optimal enzyme:substrate ratio of trypsin is about 1:10.

Digestion may be conducted at a pH of about 7, about 7.1, about 7.2, about 7.3, about 7.4, about 7.5, about 7.6, about 7.7, about 7.8, about 7.9, or about 8. In some exemplary embodiments, an optimal pH for digestion is about 7.5.

While the method described above recites the characterization of at least one disulfide bond of a protein of interest, it should be understood that this method may be extended to a variety of applications. For example, disclosed herein is a method of characterizing the complete disulfide bond structure of a protein of interest, comprising applying the method of characterizing at least one disulfide bond of a protein of interest to all disulfide bonds of a protein of interest. Characterization of these disulfide bonds may be applied, for example, for understanding of the native structure of a protein, or for identifying disulfide scrambling artifacts induced by any other process, for example protein isolation, protein purification, or recombinant protein production. This method may also be applied to, for example, comparing disulfide bond structures between at least two proteins, for example in order to compare their native structures, or to compare their respective susceptibility to disulfide bond scrambling. It is further understood that “characterizing” at least one disulfide bond may include, for example, identifying, quantifying, and/or comparing said at least one disulfide bond.

It is understood that the present invention is not limited to any of the aforesaid protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s), and any protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s) can be selected by any suitable means.

The present invention will be more fully understood by reference to the following examples. They should not, however, be construed as limiting the scope of the invention.

### EXAMPLES

### Materials.

Trifluoroacetic acid (TFA) and acetonitrile were purchased from Thermo Fisher Scientific (Rockford, Ill.). Urea, iodoacetamide (IAA), tris(2-carboxyethyl) phosphine hydrochloride (TCEP-HCl) and cystamine dihydrochloride were purchased from Sigma-Aldrich (St. Louis, Mo.). AccuMap low pH protein digestion kit and mass spectrometry grade Trypsin Platinum were purchased from Promega (Madison, Wis.). Tris-HCl buffer, pH 7.5 was obtained from Invitrogen (Carlsbad, Calif.). Purified monoclonal antibodies were produced internally by Regeneron (Tarrytown, N.Y.).

### Regular Non-Reduced Peptide Mapping Sample Preparation.

For regular non-reduced peptide mapping sample preparation, a 200 μg aliquot of each mAb sample was diluted to about 3.3 μg/μL by adding 8 M urea in 100 mM Tris-HCl solution. After sample dilution, protein concentration was measured using a NanoDrop 2000 (Thermo Scientific, MA) UV-Vis spectrophotometer. A 100 μg aliquot of each sample was alkylated with 2.5 mM iodoacetamide and incubated at 50° C. for 30 minutes in the dark. Each sample was then diluted 8 times with 100 mM Tris-HCl, pH 7.5 and digested with Trypsin Platinum at an enzyme to substrate ratio of 1:5 (w/w) at 37° C. for 3 hours. Half of the samples were transferred to another tube and reduced with 10 mM TCEP at 37° C. for one hour. Non-reduced and reduced digestions were quenched by adding TFA to a final concentration of 0.3%.

### Cystamine-Added Non-Reduced Peptide Mapping Sample Preparation.

For cystamine-added non-reduced peptide mapping sample preparation, the protocol is identical to the regular non-reduced peptide mapping method with the exception that cystamine was added into the sample preparation buffer during sample dilution. In brief, 200 μg mAb samples were buffer exchanged into 8 M urea and 1 mM cystamine dihydrochloride in 100 mM Tris HCl, pH 7.5 buffer to a final concentration of 3.3 μg/μL using 10 kDa Amicon MWCO centrifugal filters. A 100 μg aliquot of each sample was then alkylated with 2.5 mM iodoacetamide and incubated at 50° C. for 30 minutes in the dark. Each sample was then diluted about 8-fold with 100 mM Tris-HCl (pH 7.5) and 1 mM cystamine dihydrochloride followed by digestion with Trypsin Platinum at an enzyme to substrate ratio of 1:5 (w/w) at 37° C. for 3 hours. Digestion was quenched by adding TFA to a final concentration of 0.3% before LC-MS analysis.

### Low pH Non-Reduced Peptide Mapping Method.

The AccuMAP low pH protein digestion kit from Promega was used for low pH non-reduced peptide mapping sample preparation. The protocol followed the manufacturer's technical manual with minor modifications. Briefly, samples (100 μg) were diluted to ˜5 μg/μL using 8 M guanidine hydrochloride, pH ˜5.6 solution. The diluted samples were alkylated in 15 mM N-Ethylmaleimide (NEM) and incubated at 37° C. for 2 hours. The alkylated samples were pre-digested at 37° C. for 1 hour using low pH resistant rLys-C from the kit. Then, the samples were diluted five-fold with low pH reaction buffer and digested another 3 hours by adding modified trypsin and low pH resistant rLys-C following the ratio of enzyme:substrate specified by the manufacturer. Digestion was quenched by adding TFA to a final concentration of 0.3% before LC-MS analysis.

LC/UV-MS analysis.

A Waters ACQUITY UPLC I-Class system coupled to a Thermo Scientific Q Exactive Plus mass spectrometer was used to analyze the non-reduced digested samples. The tryptic peptide mixture was separated by a Waters ACQUITY UPLC BEH® 130 C18 column (1.7 μm, 2.1 mm×150 mm) at a flow rate of 0.25 mL/minute. Mobile phase A was 0.05% TFA in water and mobile phase B was 0.045% TFA in acetonitrile. The gradient was held at 0.1% B for the first 5 min and then increased to 26% B in 55 min followed by another increase to 34.5% B in 35 min. The column was equilibrated with 99.9% mobile phase A prior to sample injection, with the column temperature maintained at 40° C. The MS data were acquired on a Thermo Scientific Q Exactive Plus mass spectrometer from m/z 300-2000 at a resolution of 70 k (at m/z 400), followed by five data-dependent MS/MS scans at a resolution of 17.5 k. MS full scans were set at 1×10⁶ automated gain control (AGC) and a maximum injection time of 50 ms. MS² fragmentation was performed using HCD with a normalized collision energy of 28% at a 1×10⁵ AGC, and a maximum injection time of 100 ms. Dynamic exclusion duration was set to 15 seconds with a single repeat count.

### Data Analysis.

All peptide identity assignments and post-translational modification identifications were performed using Protein Metrics Byonic™ (version 3.11.3) by searching the raw files against the mAb protein sequence. The preliminary list of unique peptides was generated by filtering against a 1% FDR. The list of precursors and the original searching results as a spectra library were then imported into Skyline Daily software (University of Washington, WA) for a full scan (MS¹)-based final ID validation. The peak area was extracted by summing all charge states through Skyline software.

### Example 1. Identification of Abundant Disulfide Scrambled Artifacts Under Regular Digestion Conditions from mAb1 and Potential Issues Using Low pH Digestion Conditions

A human monoclonal IgG1 antibody with lambda light chains, referred to as mAb1, was used as a model protein in this study. Disulfide bond linkages on a representative IgG1 mAb structure are displayed in FIG. 1A. Cysteine amino acids are numbered based on their relative order in the sequence of HC and LC from N-terminal to C-terminal, from LC1 to LC5 in the light chains and HCl to HC11 in the heavy chains. “N” represents an N-glycosylation site. Labeled regions include the constant region of the heavy chain (CH), constant region of the light chain (CL), variable region of the heavy chain (VH), and variable region of the light chain (VL).

Non-reduced peptide mapping analysis produces distinctive disulfide peptides, each featuring an identifiable disulfide bond. Under regular non-reduced peptide mapping conditions, the eight distinguishable native IgG1 disulfide peptides were identified (for example, LC5-HC5). However, multiple disulfide scrambled peptides were also observed (for example, LC4-LC5). All IDs were identified by LC-MS and parallel TCEP-reduced experiments (data not shown). The disulfide scrambled peptides were ranked based on their percentage abundance relative to all disulfide scrambled peptides, as shown in FIG. 1B. The percentage was calculated using the peak area of each disulfide scrambled peptide compared to the sum of the peak areas from all disulfide scrambled peptides.

LC4-LC5 and HC3-HC5, two disulfide scrambled peptides resulting from the HC-LC inter-disulfide bond disruption, are the most abundant disulfide scrambled peptides and account for ˜70% of the total disulfide scrambled peptides' abundance. Therefore, these two disulfide scrambled peptides were used as performance indicators to evaluate different non-reduced peptide mapping methods.

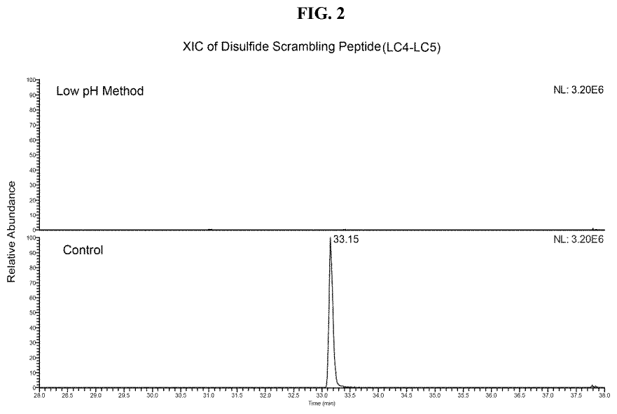

It is well known that acidic pH can effectively prevent disulfide scrambling during sample preparation (Wang et al., 2016, Anal Biochem, 495:21-8; Liu et al., 2014, Mol Cell Proteomics, 13(10):2776-86; Sung et al., 2016, Biochim Biophys Acta, 1864(9):1188-1194). To confirm that the disulfide scrambled species in the mAb1 samples were artifacts generated during sample preparation, a low pH (˜5.6) sample preparation kit from Promega was employed to prepare non-reduced digests of mAb1. As shown in FIG. 2, the most abundant disulfide scrambled peptides were not observed in the low pH condition. All other disulfide scrambled peptides identified during regular digestion conditions were either observed at negligible levels or not observed in low pH conditions, which demonstrated that these disulfide scrambled species are sample preparation-induced artifacts.

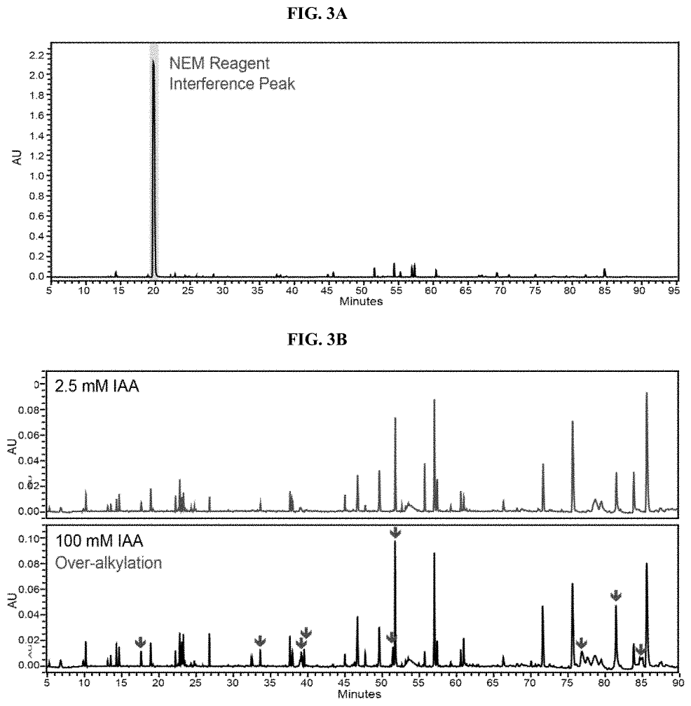

A complication from the low pH digestion method is that although the acidic pH method can effectively prevent disulfide scrambling during sample preparation, an intense interference peak at a retention time of 20 min dominates the UV chromatograms, as shown in FIG. 3A. This peak was identified to be an N-Ethylmaleimide (NEM) alkylation reagent peak by control experiments. In addition to its negative impact on the quality of UV chromatograms, this intense reagent peak could mask other tryptic peptide peaks from mAb digestion.

Additionally, since acidic pH is not ideal for most commonly used enzymes, potentially decreased digestion efficiency of the low pH method could compromise method reproducibility and precision, even when two digestive enzymes are used. Increased numbers of mis-cleaved sites in acidic pH conditions add extraneous features to chromatograms and muddle assignment of disulfide peptides. All of these adverse factors negatively impact the low pH method for UV-based peptide mapping method qualification. Therefore, a need exists for a method that can restrict disulfide scrambling during sample preparation at basic pH conditions.

### Example 2. Prevention of Disulfide Scrambling by Addition of Cystamine at Basic pH

To address the challenges described above in preventing sample preparation-induced disulfide scrambling in non-reduced peptide mapping, a simple non-reduced peptide mapping method for mAb characterization was developed that prevents disulfide scrambling at basic pH by adding cystamine. Cystamine is an oxidizing agent commonly used to promote classical disulfide bond formation during in vitro experiments (Mamathambika et al.; Huth et al., 1994, Biotechnol Bioeng, 44(1):66-72; Pompach et al., 2009, J Mass Spectrom, 44(11):1571-8). It was discovered that cystamine could be used for the novel purpose of preventing disulfide bond opening and scrambling during sample preparation for non-reduced peptide mapping analysis.

To test the degree to which addition of cystamine prevents disulfide scrambling, five different experimental conditions were designed in triplicate, using mAb1 as an exemplary protein. The regular non-reduced peptide mapping method's procedure was adapted to the five experiments, but different alkylation reagents and concentrations were used for cysteine alkylation. The five experiments used no alkylation reagent (control), 2.5 mM IAA, 100 mM IAA, 1 mM cystamine, and a combination of 2.5 mM IAA and 1 mM cystamine for alkylation, respectively. To normalize the variation among different runs, a well-separated peptide located in the heavy chain constant region without modifications or disulfide bonds was selected to normalize disulfide scrambled peptide peak areas.

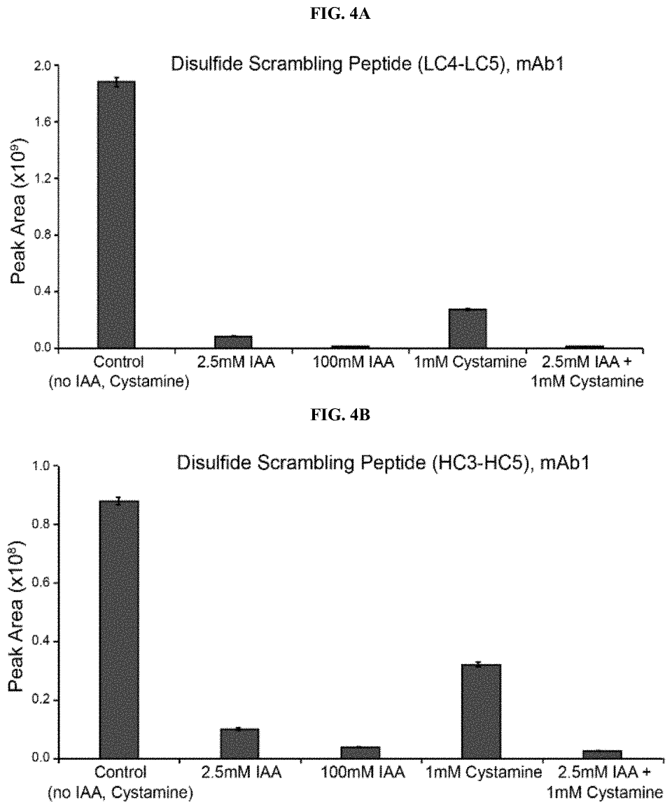

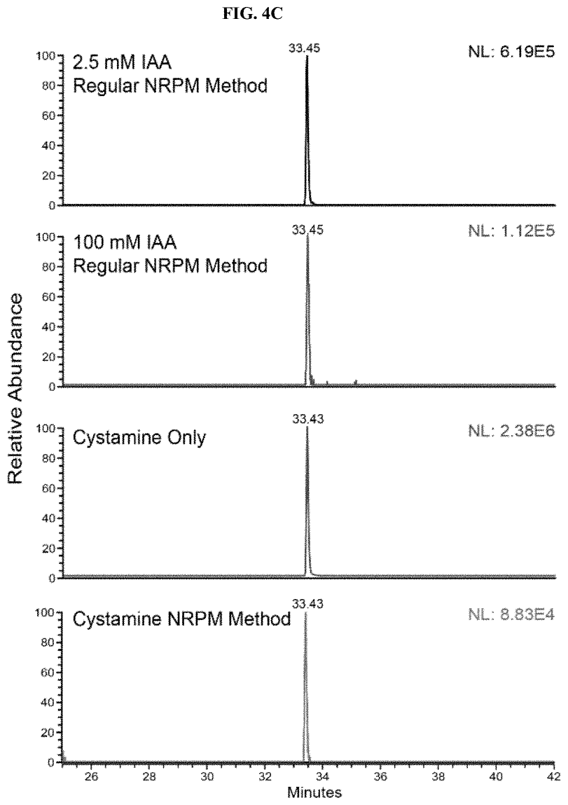

The two disulfide scrambled peptides (LC4-LC5 and HC3-HC5) that are related to HC-LC disulfide disruption were identified with high confidence by LC-MS in the control experiments and further confirmed based on their retention time and peak intensity shift during TCEP-reduced experiments. As shown in FIGS. 4A and 4B, the best results were obtained when combining 2.5 mM IAA and 1 mM cystamine (the cystamine-added method of the present invention), where the peak areas of the two disulfide scrambled peptides were reduced significantly to negligible levels compared to other methods. Addition of cystamine can prevent disulfide scrambling significantly with minor changes to the regular method. The extracted ion chromatogram's (XIC) area for the LC4-LC5 disulfide scrambled peptide decreased more than seven times when the cystamine-added non-reduced peptide mapping method was used compared to the regular method, as shown in FIG. 4C (8.83E4 compared to 6.19E5). In addition, the cystamine-added method was executed at basic pH with minor changes to the regular method, resulting in a method with high digestion efficiency, high reproducibility, and less variation. As a result, qualification of this method to monitor drug substance quality at various stages of drug development should be relatively simple.

The two disulfide scrambled peptides had much larger XIC peak areas in control and cystamine-only experiments compared to the other conditions. The observation is reasonable because endogenous free thiols were not capped, which makes the HC-LC inter-disulfide more vulnerable to disruption and eventual disulfide scrambling. In the regular method, 2.5 mM iodoacetamide (IAA) was used to alkylate all free thiols before digestion. However, the two disulfide scrambled peptides were still observable, indicating that disulfides could open to form scrambles during digestion. Since cystamine prevents disulfide disruption at basic pH during all stages of sample preparation, combining 2.5 mM IAA and 1 mM cystamine significantly minimizes disulfide scrambling before and during digestion compared to the regular method.

Significantly increasing the concentration of the alkylating reagent during sample preparation can also decrease disulfide scrambling according to other reports (Liu et al., 2007; Zhang et al., 2019). When the IAA concentration was increased to 100 mM, the peak areas of both disulfide scrambled peptides decreased multiple folds. However, an alkylating reagent like iodoacetamide at high concentration was found to generate significant levels of over-alkylation products, as shown in FIG. 3B, wherein multiple amino acid residues become undesirably modified, and over-alkylated peaks were observed in UV chromatograms (Boja and Fales; Pompach et al.). Over-alkylated peptide peaks are marked by arrows.

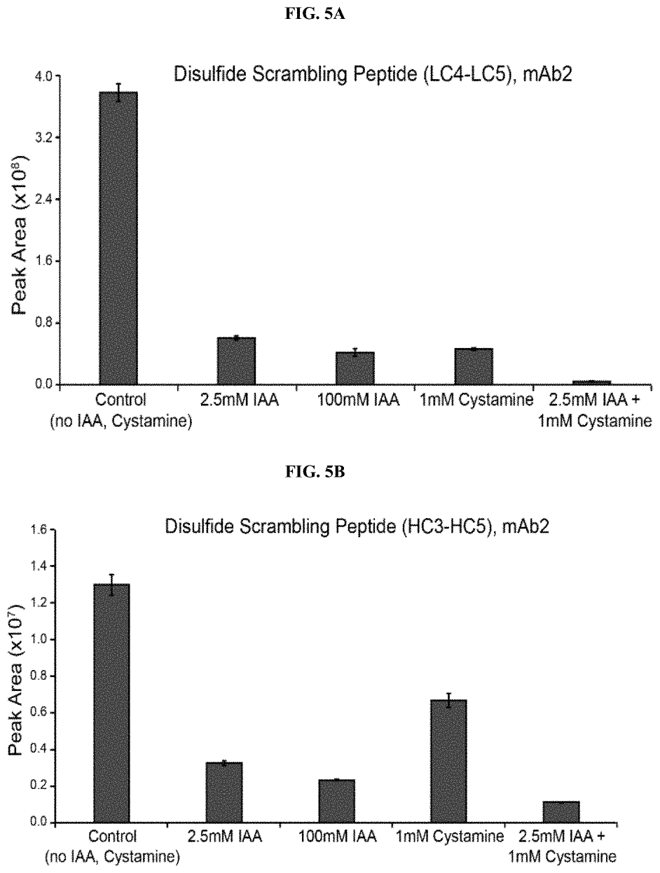

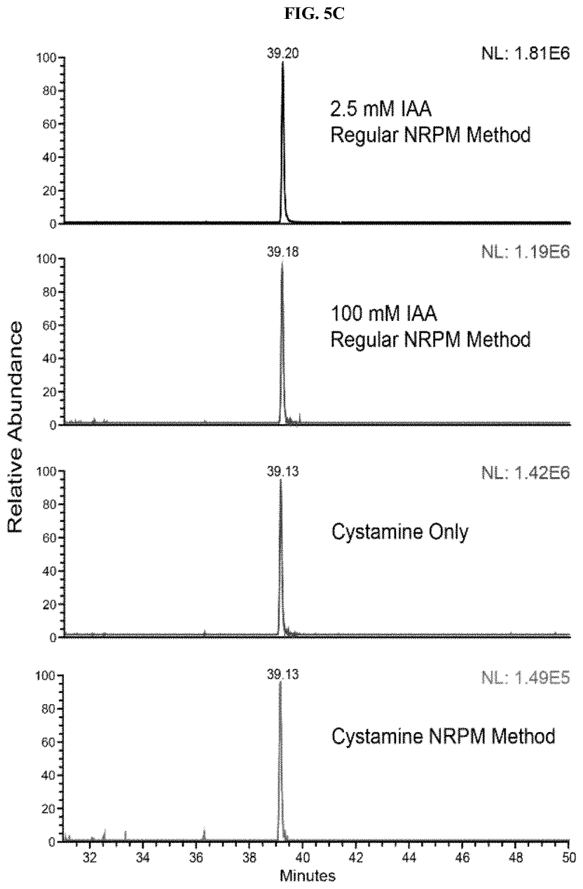

Another human IgG1 mAb, referred to herein as mAb2, has kappa light chains and was also observed to contain a high level of disulfide scrambling on cysteines involved in HC-LC inter-chain disulfide bond. To further validate the method of the present invention, the novel cystamine-added method was used to minimize disulfide scrambling of mAb2. The five different conditions described above were executed to test the disulfide scrambling level of mAb2 samples. The two disulfide scrambled peptides (LC4-LC5 and HC5-HC3) resulting from the HC-LC disulfide disruption were identified with high confidence by LC-MS. As shown in FIGS. 5A and 5B, optimal results were obtained from the cystamine-added non-reduced peptide mapping method, where the peak areas of the two disulfide scrambled peptides decreased significantly compared to other methods. The two disulfide scrambling artifacts were reduced to negligible levels when using the cystamine-added method. The XIC areas for the LC4-LC5 disulfide scrambled peptide were ten times less in maps produced from the cystamine-added non-reduced peptide mapping method compared to the regular method, as shown in FIG. 5C. These results demonstrate that the cystamine-added method can efficiently prevent sample preparation-induced disulfide scrambling artifacts in multiple different proteins, for example different subtypes of IgG1 mAb samples.

### Example 3. The Effect of Denaturation Temperature on the Cystamine-Added Method

Denaturation temperature influences digestion efficiency and disulfide scrambling. Denaturation at lower temperatures (room temperature or 37° C.) can minimize disulfide scrambling during mAb disulfide analysis, based on a previous report (Wang et al.), but complete denaturation of an antibody at low temperatures is difficult, and incomplete unfolding can decrease digestion efficiency by restricting access to trypsin cleavage sites (Cheng et al., 2016, J Pharm Biomed Anal, 129:203-209). To assess the efficacy of the method of the present invention at different temperatures, both mAb1 and mAb2 were denatured and alkylated in 8 M urea at 37° C. and 50° C. and subjected to cystamine-added non-reduced peptide mapping analysis.

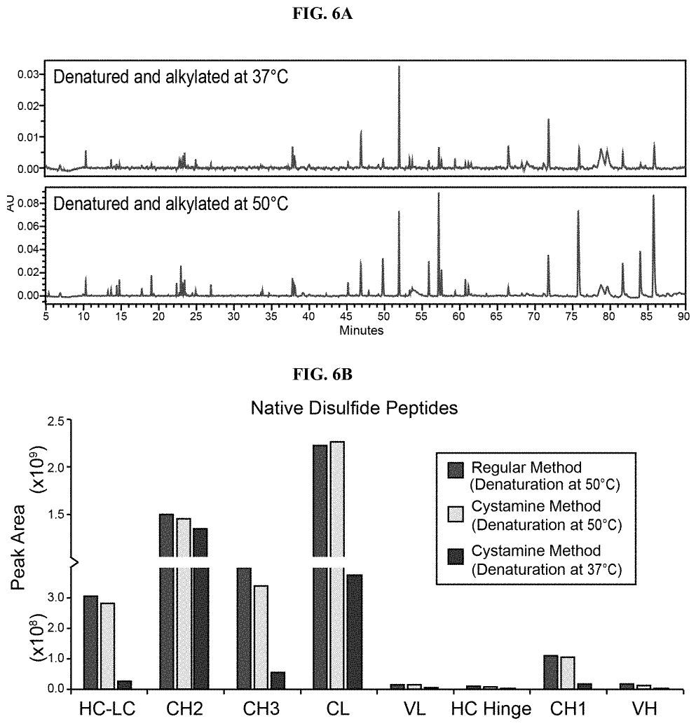

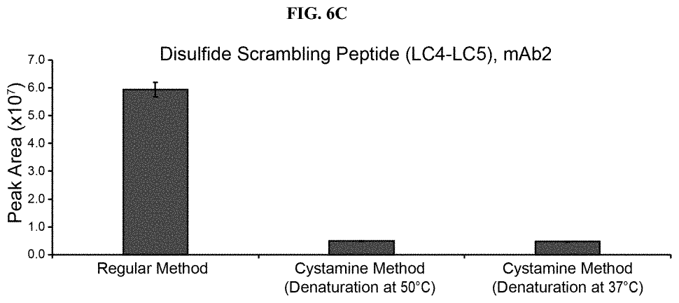

For mAb1, insufficient digestion efficiency was observed at 37° C. As shown in FIG. 6A, the sample denatured at 37° C. has fewer peptide peaks and lower overall peak intensity compared to the one at 50° C. As shown in FIG. 6B, all canonical disulfide-linked peptide peak areas were compared between the regular method with denaturation at 50° C., the cystamine-added method with denaturation at 50° C., and the cystamine-added method with denaturation at 37° C. The peak areas of classical disulfide peptides are much lower in the samples denatured at 37° C. than the samples denatured at 50° C. because of insufficient digestion efficiency in the samples denatured at 37° C. For IgG molecules like mAb1, the stable tertiary structures from 16 disulfide linkages may result in incomplete denaturation with 8 M urea at low temperatures, leading to insufficient access to cleavage sites for enzymes. Higher temperatures may be necessary to help unfold the mAb completely and consequently improve mAb digestion efficiency and reproducibility. Thus, to achieve high digestion efficiency and high reproducibility under non-reduced conditions, denaturing samples at 50° C. may be optimal for cystamine-added non-reduced peptide mapping characterization for certain proteins.

For mAb2 samples, similar UV chromatogram profiles and peak intensities (data not shown) were observed regardless of the two denaturation temperatures. Much lower disulfide scrambling levels were observed using the cystamine-added method at both denaturation temperatures compared to the regular method, as shown in FIG. 6C. These results demonstrate that the cystamine-added method prevents disulfide scrambling independent of the two selected temperatures as long as high digestion efficiency is achieved. Overall, these results demonstrate that the cystamine-added method can prevent disulfide scrambling at basic pH and high denaturation temperatures. High digestion efficiency and minimum disulfide scrambling were achieved using the cystamine-added method.

A robust sample preparation method was developed to minimize disulfide scrambling artifacts produced during sample preparation and characterize endogenous disulfide bonds. LC-MS results showed that the new method of adding cystamine can minimize disulfide scrambling significantly and has high digestion efficiency for multiple proteins, for example for two different types of IgG samples, compared with the regular method. By adding cystamine, this new method effectively prevented protocol-induced disulfide scrambling compared to the regular non-reduced peptide mapping method. Furthermore, this novel method allows for complete denaturation of mAb samples at higher temperatures in 8M urea denaturing buffer and digestion at optimal pH condition for optimal trypsin activity. Thus, high digestion efficiency was achieved, which in turn increased method robustness and reproducibility.

In contrast, most reported methods often compromise digestion efficiency by utilizing low pH or low denaturation and digestion temperatures in order to minimize disulfide scrambling (Wang et al.; Liu et al., 2014; Sung et al.; Cui et al., 2019, J Proteomics, 198:78-86). Good digestion efficiency typically increases protocol repeatability when performed by different analysts, and it also achieves higher amino acid sequence coverage and fewer missed cleavages. Incomplete digestion increases the complexity of the chromatograms and also complicates analysis, especially quantitation, because one species will be distributed into two or three cleavage products (Wang et al.). The results from testing the low pH method indicated that it could effectively prevent disulfide scrambling, but its digestion efficiency also dropped. The digestion efficiency for the low pH method could be increased by denaturing and alkylating at low pH, performing buffer exchange, and then digesting at basic pH (Nie et al., 2019, 67th ASMS). However, the dominant interference peaks produced by the alkylation reagent (NEM) in the low pH method may hinder its viability, especially for development of qualified non-reduced peptide mapping protocols characterizing therapeutic mAbs.

The oxidative compound cystamine was used to prevent disulfide bond opening during sample preparation in the non-reduced peptide mapping method of the present invention. Based on these results, high disulfide scrambling levels were observed in the cystamine-only experiments. However, combining cystamine addition with iodoacetamide alkylation resulted in the optimized cystamine-added method and a significant decrease in disulfide scrambling. This could be attributed to the different roles each reagent plays during sample preparation to prevent disulfide scrambling. In the cystamine-only experiments, free thiols were not covalently blocked, so high disulfide scrambling levels were still observed. After capping free thiols using alkylation reagents like iodoacetamide, cystamine prevents disulfide reduction and subsequent disulfide bond scrambling.

Other studies have shown that the HC-LC interchain disulfide bond in IgG1 is the most vulnerable disulfide for reduction (Liu et al., 2010, Anal Chem, 82(12):5219-26). The two IgG1 mAbs tested here have different type LCs (lambda and kappa). The two most abundant disulfide scrambled peptides in both mAbs under regular digestion conditions are related to HC-LC interchain disulfide bonds, whose reduction could lead to the formation of high levels of low molecular weight (LMW) species for both molecules. A high level of LMW species made of free light chain was observed from other assays like a microchip electrophoresis (MCE) assay (data not shown). It was also found that mAb1, which contains lambda LC, had a higher degree of disulfide scrambling on Cys 5 of both light and heavy chains and correspondingly higher LMW species than mAb2, which contains kappa LC. This observation is consistent with previous reports that IgGλ, is more susceptible to LC-HC inter-disulfide bond reduction compared to IgGκ antibody molecules (Montano and Morrison, 2002, J Immunol, 168(1):224-31; Shen et al., 2013, MAbs, 5(3):418-31).

In addition to minimizing disulfide scrambling, canonical disulfide bond identification and confirmation, LC/UV-MS compatibility, reproducibility, and disulfide bond quantification are also critical components to non-reduced peptide mapping method development for therapeutic mAb characterization. Finally, this novel method was successfully applied to two different type of IgG mAbs to prevent disulfide scrambling.

As demonstrated above, the new non-reduced peptide mapping method can significantly reduce disulfide scrambling artifacts when the mAb samples were denatured at a higher temperature and digested under basic pH. This method has been successfully used for two different types of IgG1 antibody disulfide structure characterization, where all endogenous disulfide bonds were characterized and disulfide scrambling artifacts were minimized. The results also showed that the method is robust with high digestion efficiency and LC/UV-MS compatibility. This new sample preparation method developed here together with subsequent LC-MS analysis provides the biotechnology industry with a robust approach for determining disulfide bonding pattern and assessing their integrity and stability with applications to different stages of mAb development including formulation, forced degradation, and stability studies.

What is claimed is:

1. A method for characterizing at least one disulfide bond of a protein of interest, comprising:
(a) preparing a peptide digest of a protein of interest, said preparing including:
(i) contacting a sample including a protein of interest to cystamine and to at least one denaturation agent to form a denatured protein of interest;
(ii) contacting said denatured protein of interest to an alkylation agent to form an alkylated protein of interest; and
(iii) contacting said alkylated protein of interest to a digestive enzyme to form a peptide digest;
(b) subjecting said peptide digest to analysis using liquid chromatography-mass spectrometry to identify at least one peptide that includes a disulfide bond; and
(c) using said at least one identified peptide to characterize at least one disulfide bond of said protein of interest.

2. The method of claim 1, further comprising adding cystamine to said denatured protein of interest, adding cystamine to said alkylated protein of interest, or a combination thereof.

3. The method of claim 1, wherein the concentration of cystamine is between about 0.5 mM and about 2 mM, optionally wherein the concentration of cystamine is about 1 mM.

4. The method of claim 1, further comprising comparing said at least one identified peptide to at least one identified peptide from a control sample including said protein of interest, wherein said control sample is additionally subjected to a protein reduction step.

5. The method of claim 1, wherein said protein of interest is an antibody.

6. The method of claim 5, wherein said protein of interest is a monoclonal antibody or a bispecific antibody.

7. The method of claim 1, wherein said at least one denaturation agent is urea.

8. The method of claim 7, wherein said urea is present at between about 6 M and about 10 M, optionally wherein said urea is present about 8 M.

9. The method of claim 1, wherein said denaturation is conducted at a pH between about 7 and about 8, optionally wherein said denaturation is conducted at a pH of about 7.5.

10. The method of claim 1, wherein said denaturation is conducted at about 37° C. or about 50° C.

11. The method of claim 1, wherein said alkylation agent is iodoacetamide.

12. The method of claim 11, wherein said iodoacetamide is present at between about 1 mM and about 20 mM, optionally wherein said iodoacetamide is present at about 2.5 mM.

13. The method of claim 1, wherein said alkylation is conducted at a pH between about 7 and about 8, optionally wherein said alkylation is conducted at a pH of about 7.5.

14. The method of claim 1, wherein said digestive enzyme is trypsin.

15. The method of claim 14, wherein said trypsin is present at between about a 1:5 enzyme:substrate ratio and about a 1:20 enzyme:substrate ratio, optionally wherein said trypsin is present at about a 1:10 enzyme:substrate ratio.

16. The method of claim 1, wherein said digestion is conducted at a pH between about 7 and about 8, optionally wherein said digestion is conducted at a pH of about 7.5.

17. The method of claim 1, wherein said chromatography step comprises reverse phase liquid chromatography, ion exchange chromatography, size exclusion chromatography, affinity chromatography, hydrophobic interaction chromatography, hydrophilic interaction chromatography, mixed-mode chromatography, or a combination thereof.

18. The method of claim 1, wherein said mass spectrometer is an electrospray ionization mass spectrometer, nano-electrospray ionization mass spectrometer, or an Orbitrap-based mass spectrometer, wherein said mass spectrometer is coupled to said liquid chromatography system.
